(19) United States
(12) Patent Application Publication
(10) Pub. No.: US 2005/0005935 A1
(43) Pub. Date: Jan. 13, 2005

(54) Respiratory apparatus and methods of respiratory treatment

(76) Inventor: Lewis George Gradon, Auckland (NZ)

Correspondence Address: TREXLER, BUSHNELL, GIANGIORGI,;BLACKSTONE & MARR, LTD., 105 WEST ADAMS STREET, SUITE 3600, CHICAGO, IL, 60603, US

(21) Appl. No.: 10/489,143

(22) PCT Filed: Sep. 17, 2002
(86) PCT No.: PCT/NZ02/00182

(30) Foreign Application Priority Data
Sep. 18, 2001 (NZ) 514277

(51) Int. Cl. A61M 16/00

(52) U.S. Cl. 128/204.18; 128/204.21; 128/204.23

(57) ABSTRACT

Methods and apparatus for detecting apnoea that use ultrasonic sound waves are disclosed. The method consists of applying ultrasonic sound waves of varying frequency to a patient's airway and detecting their reflected sound waves. The sound waves are then analysed to determine whether there is a narrowing of the airway. In particular a transmitting ultrasonic transducer and receiving ultrasonic transducer are located on the interior surface of a mask and positioned so as to be directed towards the nares of the patient. A signal is emitted through the transmitting transducer and received at the receiving transducer. The received signal indicates a distance to a reflection or a distance to the narrowing of the upper respiratory tract. In this manner obstructions or narrowing of a patient's airway tract may be identified as reflections which appear at anatomically appropriate distances.

### TECHNICAL FIELD

The present invention relates to a method and apparatus for detecting an apnoea (the complete cessation of breathing) and hypopnoea (decreased ventilation) during a patient's sleep and to the determination of airway patency. The invention also relates to the detection of partial obstruction of the airway (i.e. obstructed breathing), and to the treatment of sleep apnoea once an apnoea is detected.

### BACKGROUND ART

Obstructive Sleep Apnoea (OSA) is a sleep disorder which affects up to at least 5% of the population in which muscles which normally hold the airway open relax and ultimately collapse, sealing the airway. The sleep pattern of an OSA sufferer is characterised by repeated sequences of snoring, breathing difficulty, lack of breathing, waking with a start and then returning to sleep. Often the sufferer is unaware of this pattern occurring. Sufferers of OSA usually experience daytime drowsiness and irritability due to a lack of good continuous sleep.

In an effort to treat OSA sufferers, a technique known as Continuous Positive Airway Pressure (CPAP) was devised. A CPAP device consists of a gases supply (or blower) with a conduit connected to supply pressurised gases to a patient, usually through a nasal mask. The pressurised air supplied to the patient effectively assists the muscles to keep the patient's airway open, eliminating the typical OSA sleep pattern.

The expression “airway” as used herein is to be understood as the anatomical portion of the respiratory system between the nares and the bronchi, including the trachea and oral cavity. The expression “respiration” is to be understood as the continually repeating events of inspiration (inhaling) followed by expiration (exhaling).

The expression “narrowing” includes partial and total occlusion when used to refer to the narrowing of a patient's airway.

In sleep apnoea syndrome a person stops breathing during sleep. Cessation of airflow for more than 10 seconds is called an “apnoea”. Apnoeas lead to decreased blood oxygenation and thus to disruption of sleep. Apnoeas are traditionally categorized as either central, where there is no respiratory effort, or obstructive, where there is respiratory effort. With some central apnoeas, the airway is patent, and the subject is merely not attempting to breathe. Conversely, some apnoeas may be a mixture of central apnoeas followed by an obstructive event.

The airway may also be partially obstructed (i.e. narrowed or partially patent). This also leads to decreased ventilation (hypopnoea), decreased blood oxygenation and disturbed sleep.

The dangers of obstructed breathing during sleep are well known in relation to the Obstructive Sleep Apnoea (OSA) syndrome. Apnoea, hypopnoea and heavy snoring are recognised as causes of sleep disruption and risk factors in certain types of heart disease. More recently it has been found that increased upper airway resistance (Upper Airway Resistance syndrome) during sleep without snoring or sleep apnoea also can cause sleep fragmentation and daytime sleepiness. It is possible there is an evolution from upper airway resistance syndrome to sleep apnoea, accompanied by a worsening of clinical symptoms and damage to the cardiovascular system.

The common form of treatment of these syndromes is the administering of Continuous Positive Airway Pressure (CPAP). The procedure for administering CPAP treatment has been well documented in both the technical and patent literature. Briefly stated, CPAP treatment acts as a pneumatic splint of the airway by the provision of a positive pressure, usually in the range 4 to 20 cm H₂O. The air is supplied to the airway by a motor driven blower whose outlet passes via an air delivery hose to a nose (or nose and/or mouth) mask sealingly engaged to a patient's face. An exhaust port is provided in the delivery tube proximate to the mask. More sophisticated forms of positive airway pressure devices, such as bi-level devices and auto-titrating devices, are described in U.S. Pat. No. 5,148,802 of Respironics, Inc. and U.S. Pat. No. 5,245,995 of Rescare Limited, respectively.

Various techniques are known for sensing and detecting abnormal breathing patterns indicative of obstructed breathing. U.S. Pat. No. 5,245,995, for example, describes how snoring and abnormal breathing patterns can be detected by inspiration and expiration pressure measurements while sleeping, thereby leading to early indication of preobstructive episodes or other forms of breathing disorder. Particularly, patterns of respiratory parameters are monitored, and CPAP pressure is raised on the detection of predefined patterns to provide increased airway pressure to, ideally, subvert the occurrence of the obstructive episodes and the other forms of breathing disorder.

As noted above, central apnoeas need not involve an obstruction of the airway, and often occur in patients with various cardiac, cerebrovascular and endocrine conditions unrelated to the state of the upper airway. In those cases where the apnoea is occurring without obstruction of the airway, there is little benefit in treating the condition by techniques such as CPAP. Also, known automated CPAP systems cannot reliably distinguish central apnoeas with an open airway from apnoeas with a closed airway, and may inappropriately seek to increase the CPAP splinting air pressure unnecessarily. Such unnecessary increases in pressure reflexively inhibit breathing, further aggravating the breathing disorder. Alternatively, as known automated CPAP systems cannot reliably distinguish between central apnoeas with an open airway and central apnoeas with an obstructed or narrowed airway treatment of the apnoea may be limited and some apnoeas not treated at all.

Other limitations associated with the prior art include the inability to detect airway patency and the absence of progressive response to increasingly severe indicators of airway obstruction for which the mask pressure should be increased.

### SUMMARY OF THE INVENTION

The object of the invention is to provide a method and apparatus for detecting apnoea which overcomes the abovementioned disadvantages or to at least provide the public with a useful choice.

Accordingly in a first aspect the present invention consists in a method of determining apnoea and/or hypopnoea in a patient comprising the steps of:
a) applying ultrasonic sound waves of varying frequency to a patient's airway,
b) detecting said sound waves reflected from said patients' airway,
c) analysing said reflected sound waves to determine whether there is narrowing of said airway.

In a second aspect the present invention consists in a method of determining the occurrence of apnoea and/or hypopnoea in a patient receiving continuous positive airway pressure treatment by apparatus for supplying pressurised gases to a patient, said method comprising the steps of:
a) applying ultrasonic sound waves of varying frequency to a patient's airway,
b) detecting said sound waves reflected from said patients' airway,
c) analysing said reflected sound waves to determine whether there is narrowing of said airway, and if so,
d) increasing the pressure of said gases supplied to said patient, or if not,
e) decreasing said pressure of said gases supplied to said patient.

In a third aspect the present invention consists in a method of diagnosing the narrowing of a patient's airways comprising the steps of:
a) applying ultrasonic sound waves of varying frequency to a patient's airway,
b) detecting said sound waves reflected from said patients' airway,
c) analysing said reflected sound waves to determine whether there is narrowing of said airway.

In a fourth aspect the present invention consists in apparatus for the detection of the occurrence of apnoea and/or hypopnoea in a patient comprising or including:
a) interface means providing access to said patients' airways,
b) transmitting means for transmitting ultrasonic sound waves of a varying frequency to said patients' airways,
c) receiving means for receiving the reflected sound waves from said patients' airways, and
d) data processing means for analysing said reflected sound waves to determine whether there is narrowing of said patients' airway.

In a fifth aspect the present invention consists in apparatus for the provision of CPAP treatment to the airways of a patient, comprising:
a) breathing assistance apparatus adapted to deliver gases to a patient to assist said patients' breathing including gases supply means,
b) interface means providing access to said patients' airways,
c) a conduit in fluid communication with said interface means and said breathing assistance apparatus,
c) transmitting means for transmitting ultrasonic sound waves of a varying frequency to said patients' airways,
d) receiving means for receiving the reflected sound waves from said patients' airways, and
e) data processing means for analysing said reflected sound waves to determine whether there is narrowing of said patients' airway,
f) controlling means for increasing the pressure of said gases supplied to said patient.
wherein if the narrowing of said airway is detected said controlling means increasing the pressure of said gases supplied to said patient, and if no narrowing is detected said controlling means decreasing the pressure of said gases supplied to said patient.

In a sixth aspect the present invention consists in apparatus for diagnosing the narrowing of the airways of a patient, comprising:
a) interface means providing access to said patients' airways,
b) transmitting means for transmitting ultrasonic sound waves of a varying frequency to said patients' airways,
d) receiving means for receiving the reflected sound waves from said patients' airways, and
e) data processing means for analysing said reflected sound waves to determine whether there is narrowing of said patients' airway.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

The invention consists in the foregoing and also envisages constructions of which the following gives examples.

### BRIEF DESCRIPTION OF DRAWINGS

Preferred forms of the invention will be described with reference to the accompanying drawings in which;

FIG. 1 is a diagram of a humidified continuous positive airway pressure system as might be used in conjunction with the present invention,

FIG. 2 is a diagram of the preferred form of the apparatus of the present invention for detecting apnoea, showing the use of ultrasonic transducers to detect the occurrence of narrowing of a patient's airway,

FIG. 3 is a graph of the output from the detection apparatus of the present invention when no narrowing of a patient's airway tract has been detected,

FIG. 4 is a graph of the output from the detection apparatus of the present invention when the narrowing in a patient's airway tract has been detected,

FIG. 5 is a graph showing the occluded and unoccluded output's superimposed upon one another,

FIG. 6 is an example of an algorithm that may be used for the control of the CPAP system used to treat patients having sleep apnoea syndrome, and

FIG. 7 is a further example of an algorithm that may be used for the control of the CPAP system used to treat patients having sleep apnoea syndrome.

### BEST MODES FOR CARRYING OUT THE INVENTION

It will be appreciated that the method and apparatus for detecting apnoea as described in the preferred embodiment of the present invention can be used in respiratory care generally or with a ventilator but will now be described below with reference to its use in a humidified Continuous Positive Airway Pressure (CPAP) system. CPAP System

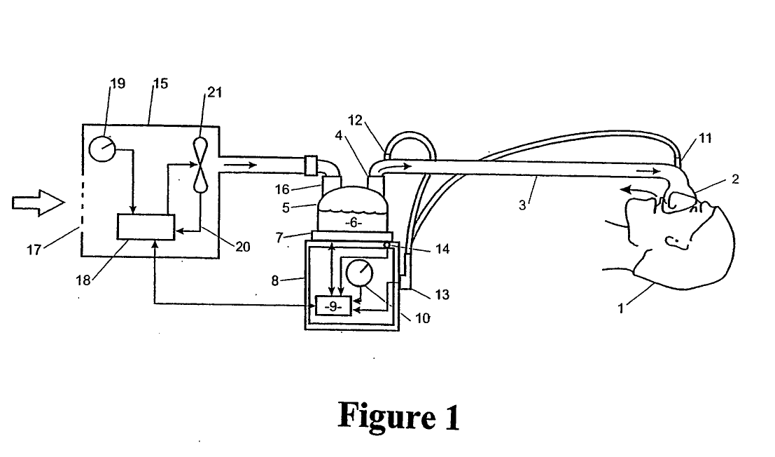

With reference to FIG. 1 a CPAP system is shown in which a patient 1 is receiving humidified and pressurised gases through a nasal mask 2 connected to a humidified gases transportation pathway or inspiratory conduit 3. It should be understood that delivery systems could also be VPAP (Variable Positive Airway Pressure) and BIPAP (Bi-level Positive Airway Pressure) or numerous other forms of respiratory therapy. Inspiratory conduit 3 is connected to the outlet 4 of a humidification chamber 5 which contains a volume of water 6. Inspiratory conduit 3 may contain heating means or heater wires (not shown) which heat the walls of the conduit to reduce condensation of humidified gases within the conduit. Humidification chamber 6 is preferably formed from a plastics material and may have a highly heat conductive base (for example an aluminium base) which is in direct contact with a heater plate 7 of humidifier 8. Humidifier 8 is provided with control means or electronic controller 9 which may comprise a microprocessor based controller executing computer software commands stored in associated memory.

Controller 9 receives input from sources such as user input means or dial 10 through which a user of the device may, for example, set a predetermined required value (preset value) of humidity or temperature of the gases supplied to patient 1. The controller may also receive input from other sources, for example temperature and/or flow velocity sensors 11 and 12 through connector 13 and heater plate temperature sensor 14. In response to the user set humidity or temperature value input via dial 10 and the other inputs, controller 9 determines when (or to what level) to energise heater plate 7 to heat the water 6 within humidification chamber 5. As the volume of water 6 within humidification chamber 5 is heated, water vapour begins to fill the volume of the chamber above the water's surface and is passed out of the humidification chamber 5 outlet 4 with the flow of gases (for example air) provided from a gases supply means or blower 15 which enters the chamber through inlet 16. Exhaled gases from the patient are exhausted to the ambient surroundings.

Blower 15 is provided with variable pressure regulating means or variable speed fan 21 which draws air or other gases through blower inlet 17. The speed of variable speed fan 21 is controlled by electronic controller 18 (or alternatively the function of controller 18 could be carried out by controller 9) in response to inputs from controller 9 and a user set predetermined required value (preset value) of pressure or fan speed via dial 19.

Detection of Apnoea

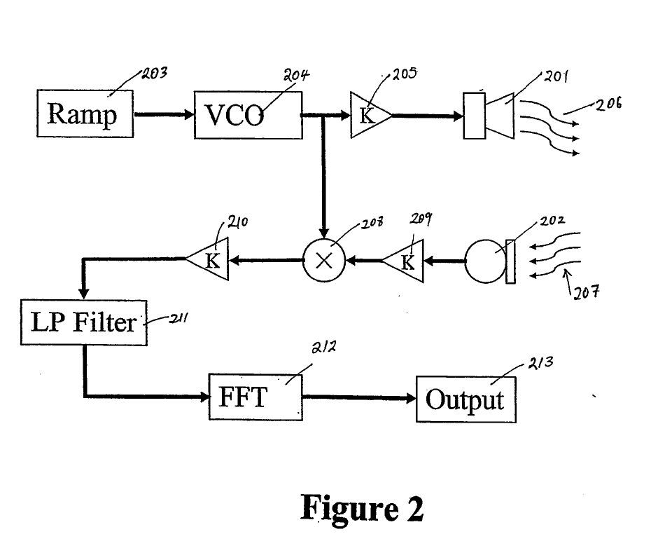

In the preferred form the method and apparatus for detecting apnoea consists of ultrasonic transducers which are used to effectively dynamically map upper airway dimensions to the extent that apnoeas may be detected in a manner which is useful to a CPAP machine or ventilation system as described above that may be used for treating obstructive sleep apnoea. Knowing that airways are narrowing and decreased ventilation, that is, hypopnoea, is occurring, the CPAP machine may increase its pressure. The transducers that determine whether there is narrowing of a patient's airways is occurring will now be described. Referring to FIG. 2, a transmitting ultrasonic transducer 201 and a receiving ultrasonic transducer 202 are located on the interior surface of a nasal mask (such as that described with reference to FIG. 1) and positioned so as to be directed towards the nares of the patient. A ramped voltage signal 203, preferably a sawtooth voltage signal as generated by control electronics and microprocessors, is fed into a voltage controlled oscillator 204 such that the output of the voltage controlled oscillator 204 sweeps from 30 kHz to 40 kHz over 1 second continuously. The continuous output signal from the voltage controlled oscillator 204 is fed through an amplifier 205 to the transmitting transducer 201. This transducer 201 then emits a variable signal 206. The ultrasonic signal is transmitted through the patient's airways and will be reflected from various structures within the upper respiratory tract and back to the receiving transducer 202 that receives the reflected signal 207. The signal transmitted from the voltage controlled oscillator 204 is also fed to a multiplier 208 where it is multiplied by the received signal 207 that has previously been amplified by a second amplifier 209. When the transmitted signal is multiplied by the received signal, the frequency of the received signal becomes dependent on the time taken for the signal to travel from the transmitter 201 to the receiver 202. Therefore when the received signal 207 is multiplied by the transmitted signal beat frequencies result, which are dependent on the distance to reflection.

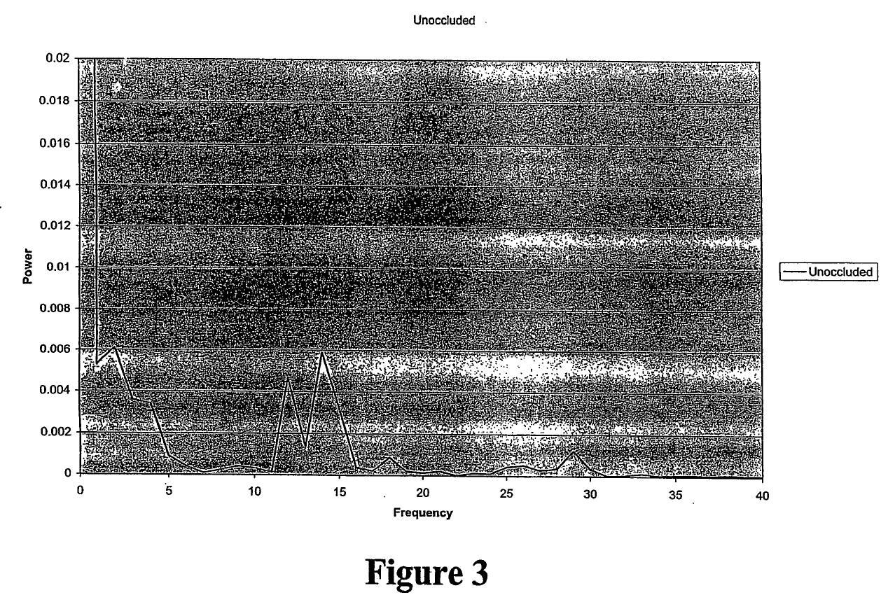

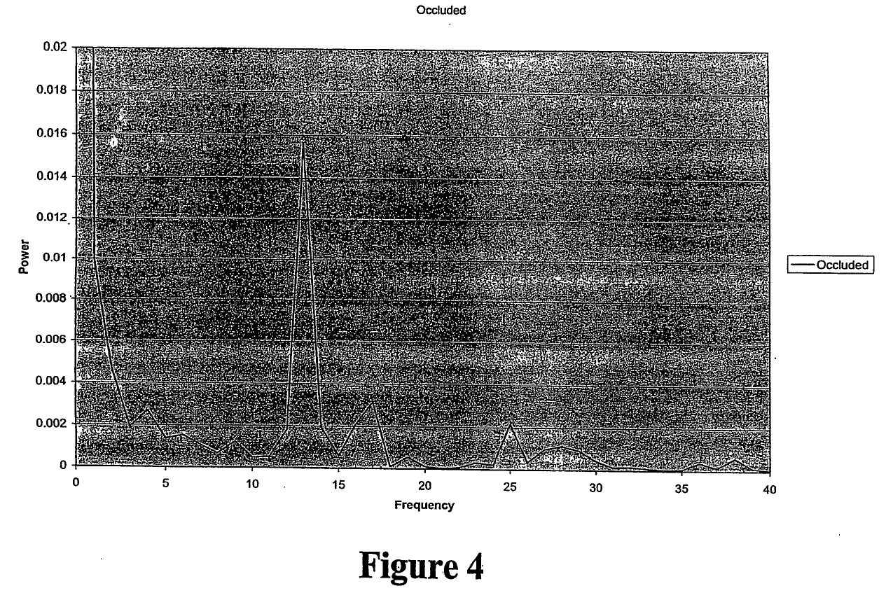

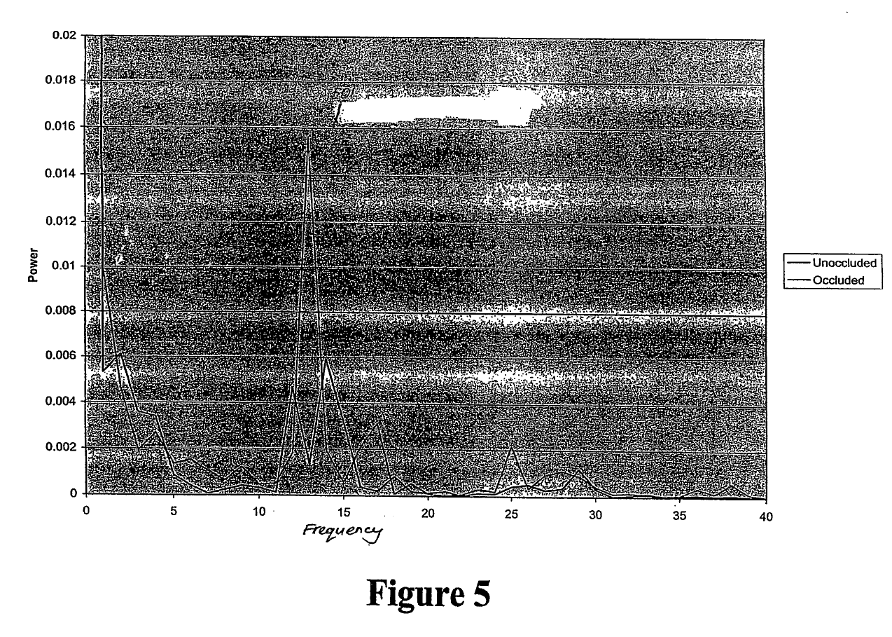

The output of the multiplier 208, namely the combined beat signal having low frequency and high frequency components, is then amplified by a further amplifier 210 and passed through a low pass filter 211. The low pass filter filters out the high frequency component of the beat signal. The filtered signal is then passed through a Fast Fourier Transform (FFT) algorithm 212. The output 213 of the FFT algorithm is a graph of the power spectrum (the mean square amplitude of a sound wave) as a function of frequency. High power output at a particular frequency corresponds to a particular reflection so the x-axis of this graph, the power spectrum, may be calibrated as the distance to the reflection, that is the distance to the narrowing of the upper respiratory tract. As can be seen in FIGS. 3 an example of the output of the FFT algorithm when the patient's airway is unoccluded is shown. Conversely, FIG. 4 shows the output of the FFT algorithm when the patient's airway is occluded. Here, the narrowing in the tract is indicated by the spike 401, and the distance along the respiratory tract can be determined by the distance that is equivalent to a power of 0.016. FIG. 5 shows the graphs of FIGS. 3 and 4 superimposed upon one another, highlighting the spike 501 where the narrowing in the patient's respiratory tract is occurring.

In this manner obstructions or the narrowing of a patient's airway tract may be identified as reflections which appear in anatomically appropriate distances. Use of a flow signal as determined by the flow velocity sensors 12, (as described with reference to FIG. 1) which gives an indication of normal breathing, can be used to assist in characterises these reflections.

Furthermore, for an obstructive apnoea the reflections from the obstruction appear when the flow signal indicates no breathing and the reflections are absent when the flow signal indicates normal breathing. Therefore, when an obstruction is detected the pressure supplied by the CPAP system increases to further assist the muscles in the patient's respiratory tract to keep the patient's airway open. When the obstruction is cleared, the apparatus of the present invention will detect this and subsequently the pressure of the air flow supplied by the CPAP system to the patient will be reduced.

For a non obstructive apnoea (central apnoea) the reflections do not change when the flow signal indicates either normal breathing or when breathing stops, thus the CPAP system will be controlled so that the pressure of the air flow supplied to the patient is not increased.

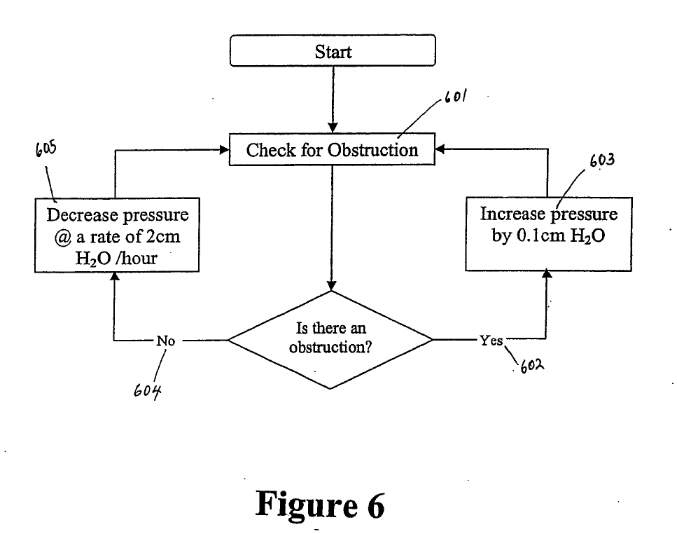

As briefly mentioned above, pressure and flow information as detected by the CPAP system may contribute to the CPAP treatment received by the patient. One example a control algorithm used by the CPAP system to control the supply of gases to the patient is illustrated in FIG. 6. This algorithm is preferably included in the control electronics and software of the CPAP system. The algorithm as shown in FIG. 6 starts first by checking if there is an obstruction 601 by using the detecting apparatus of the present invention. Next, if there is an obstruction 602 then the pressure of the airflow to the patient is increased by 0.1 cm H₂O (or any other appropriate value) 603. Alternatively, if there is no obstruction 604 the pressure of the flow to the patient is decreased at a rate of 2 cm H₂O per hour (or any other appropriate value) 605. Once the pressure of the flow is increased or alternatively decreased the algorithm again checks for the existence of an obstruction in the patient's airway. Again, depending on whether the obstruction still exists results in either the increasing or decreasing of the pressure of the flow to the patient. In this way a varying pressure provides a more efficient treatment of sleep apnoea. Furthermore, varying the pressure supplied to the patient's airways ensures the minimum pressure is delivered to the patient therefore reducing the discomfort caused by high pressures being supplied to the patient.

The apparatus of the present invention also has the advantage that it can diagnose or measure an obstruction in, or narrowing of, a patient's airway tract and treat the obstruction or narrowing accordingly. Thus this apparatus provides a definitive diagnosis of an obstruction in or narrowing of a patient's airway tract, where prior art devices only inferred from air flow measurements and the like that there is an obstruction.

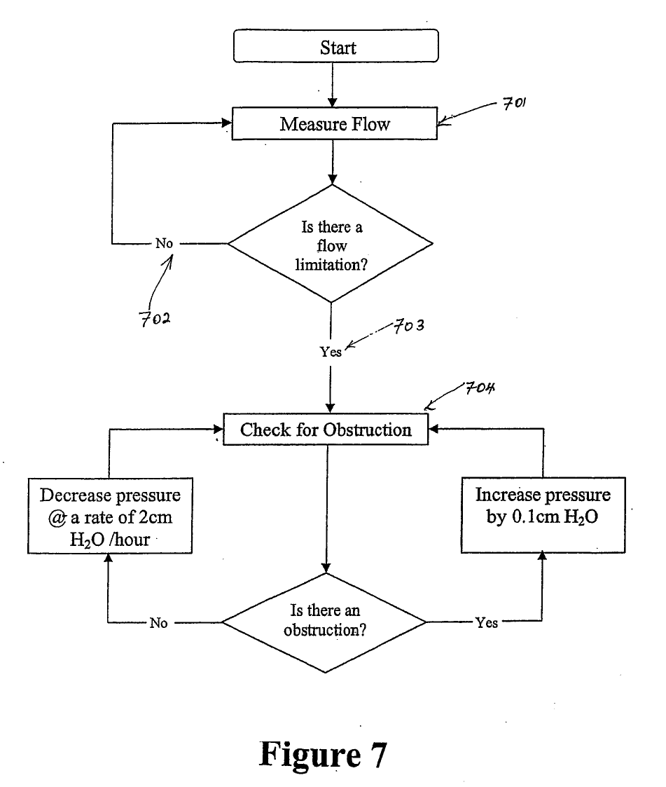

A further example of an algorithm that may be used to control the treatment of a patient using a CPAP system and the detecting apparatus of the present invention is shown in FIG. 7. Here, the algorithm uses the flow sensors as supplied with standard CPAP equipment. Firstly, the flow is measured 701 using flow sensors, and if there is no flow limitation 702, the flow is measured again, until there is a limitation 703. Then the algorithm checks for an obstruction 704, and increases or decreases the pressure of the airflow to the patient depending on whether an obstruction has been detected or not. Thus, this algorithm then follows the process of the algorithm as described above with reference to FIG. 6.

It must be appreciated that the transducers located in the mask directing the ultrasonic sound waves through a patient's nares is the preferred embodiment of the invention. Alternatively, the ultrasonic transducers may be located elsewhere, for example, in other parts of the mask, such as on the exterior, or may be at the machine end of the tubing 3 of the CPAP system as described with reference to FIG. 1. Furthermore, where the ultrasonic transducers are located at a distance from the nasal mask waveguides (small tubes) from the mask to the transducers, that have the purpose of directing the ultrasonic sound to the patient's nares or mouth, may be provided.

In an alternative embodiment of the invention there may be provided with two ultrasonic transducers, where each can transmit and receive signals. This would allow for each transducer to transmit and receive signals through alternate nostril combinations to determine the best signal. Here, each signal transmitted and received would be monitored by sweeping from each transducer, then the largest monitored signal would be used to diagnose narrowing of the airway tract.

In a further alternative embodiment there may be provided two ultrasonic transducers at each nostril, where each may either transmit or receive signals. In this way, as above, monitoring each of the signals from each of the transducers would enable the largest signal to be used to diagnose narrowing of the airway tract. Therefore, in effect choosing the largest signal will result in a signal that is least affected by noise and will provide the most accurate diagnosis.

Different combinations of software and hardware implementations of the system described here may be possible. For example, the FFT analysis may be performed by either hardware in the form of a digital signal processing (DSP) chip, or by software on a microprocessor or PC.

In the preferred form of the present invention all of the electronics making up the apparatus of the present invention, such as the voltage controlled oscillator, multiplier and low pass filter, are located at the CPAP machine end along with the control electronics controlling the operation of the CPAP machine. The exceptions to this are the transducers and amplifiers which are preferably located nearer to the patient, for example, within a nasal mask or at the oral mouthpiece.

1. A method of determining apnoea and/or hypopnoea in a patient comprising the steps of:
a) applying ultrasonic sound waves of varying frequency to a patient's airway,
b) detecting said sound waves reflected from said patient's airway,
c) analysing said reflected sound waves to determine whether there is narrowing of said airway.

2. A method of determining apnoea and/or hypopnoea in a patient according to claim 1 wherein said method includes the step of filtering said reflected sound waves.

3. A method of determining apnoea and/or hypopnea in a patient according to claims 1 or 2 wherein said step of analysing said reflected sound waves includes performing a fast Fourier transform on said reflected sound waves thereby producing an output graph of the power spectrum as a function of frequency of said reflected sound waves.

4. A method of determining apnoea and/or hypopnea in a patient according to any one of claims 1 to 3 wherein said step of analysing said reflected sound waves includes the determination of the position of said narrowing of said patient's airway by calibrating said power spectrum as the distance along said airway.

5. A method of determining the occurrence of apnoea and/or hypopnoea in a patient receiving continuous positive airway pressure treatment by apparatus for supplying pressurised gases to a patient, said method comprising the steps of:
a) applying ultrasonic sound waves of varying frequency to a patient's airway,
b) detecting said sound waves reflected from said patient's airway,
c) analysing said reflected sound waves to determine whether there is narrowing of said airway, and if so,
d) increasing the pressure of said gases supplied to said patient, or if not,
e) decreasing said pressure of said gases supplied to said patient.

6. A method of determining apnoea and/or hypopnea in a patient according to claim 5 wherein said method includes the step of filtering said reflected sound waves.

7. A method of determining apnoea and/or hypopnea in a patient according to claim 5 or 6 wherein said step of analysing said reflected sound waves includes performing a fast Fourier transform on said reflected sound waves thereby producing an output graph of the power spectrum as a function of frequency of said reflected sound waves.

8. A method of determining apnoea and/or hypopnea in a patient according to claim 5 to 7 wherein said step of analysing said reflected sound waves includes the determination of the position of said narrowing of said patient's airway by calibrating said power spectrum as the distance along said airway.

9. A method of diagnosing the narrowing of a patient's airway comprising the steps of:
a) applying ultrasonic sound waves of varying frequency to a patient's airway,
b) detecting said sound waves reflected from said patient's airway,
c) analysing said reflected sound waves to determine whether there is narrowing of said airway.

10. A method of diagnosing the narrowing of a patient's airway according to claim 9 wherein said method of diagnosing the narrowing of a patient's airway includes the step of filtering said reflected sound waves.

11. A method of diagnosing the narrowing of a patient's airway according to any one of claims 9 or 10 wherein said step of analysing said reflected sound waves includes performing a fast Fourier transform on said reflected sound waves thereby producing an output graph of the power spectrum as a function of frequency of said reflected sound waves.

12. A method of diagnosing the narrowing of a patient's airway according to any one of claims 9 to 11 wherein said step of analysing said reflected sound waves includes the determination of the position of said narrowing of said patient's airway by calibrating said power spectrum as the distance along said airway.

13. Apparatus for the detection of the occurrence of apnoea and/or hypopnoea in a patient comprising or including:
a) interface means providing access to said patient's airways,
b) transmitting means for transmitting ultrasonic sound waves of a varying frequency to said patient's airways,
c) receiving means for receiving the reflected sound waves from said patient's airways, and
d) data processing means for analysing said reflected sound waves to determine whether there is narrowing of said patient's airway.

14. Apparatus for the detection of the occurrence of apnoea and/or hypopnea in a patient according to claim 13 wherein said ultrasonic sound waves have a frequency of between 30 kHz and 40 kHz.

15. Apparatus for the detection of the occurrence of apnoea and/or hypopnoea in a patient according to any one of claims 13 or 14 wherein said interface means is a nasal mask.

16. Apparatus for the detection of the occurrence of apnoea and/or hypopnoea in a patient according to any one of claims 13 or 14 wherein said interface means is an oral mouthpiece.

17. Apparatus for the detection of the occurrence of apnoea and/or hypopnoea in a patient according to claim 15 wherein said transmitting means is at least one ultrasonic transducer located on the interior surface of said nasal mask and directed to transmit said ultrasonic sound waves into at least one of the nares of said patient.

18. Apparatus for the detection of the occurrence of apnoea and/or hypopnoea in a patient according to claim 16 wherein said transmitting means is at least one ultrasonic transducer located on or near to said oral mouthpiece and directed to transmit said ultrasonic sound waves into said patient's oral cavity.

19. Apparatus for the detection of the occurrence of apnoea and/or hypopnoea in a patient according to any one of claims 15 or 17 wherein said receiving means is an ultrasonic transducer located on the interior surface of said nasal mask and directed to receive said reflected sound waves from at least one of said nares of said patient.

20. Apparatus for the detection of the occurrence of apnoea and/or hypopnoea in a patient according to any one of claims 16 or 18 wherein said receiving means is at least one ultrasonic transducer located on or near to said oral mouthpiece and directed to received said reflected sound waves from said patient's oral cavity.

21. Apparatus for the provision of CPAP treatment to the airways of a patient, comprising:
a) breathing assistance apparatus adapted to deliver gases to a patient to assist said patient's breathing including gases supply means,
b) interface means providing access to said patient's airways,
c) a conduit in fluid communication with said interface means and said breathing assistance apparatus,
c) transmitting means for transmitting ultrasonic sound waves of a varying frequency to said patient's airways,
d) receiving means for receiving the reflected sound waves from said patient's airways, and
e) data processing means for analysing said reflected sound waves to determine whether there is narrowing of said patient's airway,
f) controlling means for increasing the pressure of said gases supplied to said patient.
wherein if the narrowing of said airway is detected said controlling means increasing the pressure of said gases supplied to said patient, and if no narrowing is detected said controlling means decreasing the pressure of said gases supplied to said patient.

22. Apparatus for the provision of CPAP treatment to the airways of a patient according to claim 21 wherein said ultrasonic sound waves have a frequency of between 30 kHz and 40 kHz.

23. Apparatus for the provision of CPAP treatment to the airways of a patient according to any one of claims 21 or 22 wherein said apparatus includes a flow sensor in fluid communication with the entrance to said patient's airways to output a signal representative of respiratory airflow of said patient as a function of time,
wherein said output signal may be used to determine if there is a limitation of the airflow to or from said patient's airway.

24. Apparatus for the provision of CPAP treatment to the airways of a patient according to any one of claims 21 to 23 wherein said interface means is a nasal mask.

25. Apparatus for the provision of CPAP treatment to the airways of a patient according to any one of claims 21 to 23 wherein said interface means is an oral mouthpiece.

26. Apparatus for the provision of CPAP treatment to the airways of a patient according to claim 24 wherein said transmitting means is an ultrasonic transducer located on the interior surface of said nasal mask and directed to transmit said ultrasonic sound waves into at least one of the nares of said patient.

27. Apparatus for the provision of CPAP treatment to the airways of a patient according to any one of claim 25 wherein said transmitting means is at least one ultrasonic transducer located on or near to said oral mouthpiece and directed to transmit said ultrasonic sound waves into said patient's oral cavity.

28. Apparatus for the provision of CPAP treatment to the airways of a patient according to any one of claims 24 or 26 wherein said receiving means is an ultrasonic transducer located on the interior surface of said nasal mask and directed to receive said reflected sound waves from at least one of said nares of said patient.

29. Apparatus for the provision of CPAP treatment to the airways of a patient according to any one of claims 25 or 27 wherein said receiving means is at least one ultrasonic transducer located on or near to said oral mouthpiece and directed to received said reflected sound waves from said patient's oral cavity.

30. Apparatus for diagnosing the narrowing of the airways of a patient, comprising:
a) interface means providing access to said patient's airways,
b) transmitting means for transmitting ultrasonic sound waves of a varying frequency to said patient's airways,
d) receiving means for receiving the reflected sound waves from said patient's airways, and
e) data processing means for analysing said reflected sound waves to determine whether there is narrowing of said patient's airway.

31. Apparatus for diagnosing the narrowing of the airways of a patient according to claim 30 wherein said ultrasonic sound waves have a frequency of between 30 kHz and 40 kHz.

32. Apparatus for diagnosing the narrowing of the airways of a patient according to any one of claims 30 or 31 wherein said interface means is a nasal mask.

33. Apparatus for diagnosing the narrowing of the airways of a patient according to any one of claims 30 or 31 wherein said interface means is an oral mouthpiece.

34. Apparatus for diagnosing the narrowing of the airways of a patient according to any one of claims 30 or 31 wherein said interface means is tubing and at least one nasal cannula locatable in at least one of said patient's nares.

35. Apparatus for diagnosing the narrowing of the airways of a patient according to claim 34 wherein said at least one nasal cannula is connected to one of said receiving means, transmitting means and data processing means by way of said tubing.

36. Apparatus for diagnosing the narrowing of the airways of a patient according to any one of claims 34 or 35 wherein said at least one nasal cannula has disposed at it's end one of said transmitting means and receiving means, said transmitting means and receiving means directed to transmit and receive sound waves into at least one of the nares of said patient.

37. Apparatus for diagnosing the narrowing of the airways of a patient according to claim 32 wherein said transmitting means is an ultrasonic transducer located on the interior surface of said nasal mask and directed to transmit said ultrasonic sound waves into at least one of the nares of said patient.

38. Apparatus for diagnosing the narrowing of the airways of a patient according to claim 33 wherein said transmitting means is at least one ultrasonic transducer located on or near to said oral mouthpiece and directed to transmit said ultrasonic sound waves into said patient's oral cavity.

39. Apparatus for diagnosing the narrowing of the airways of a patient according to any one of claims 32 or 37 wherein said receiving means is an ultrasonic transducer located on the interior surface of said nasal mask and directed to receive said reflected sound waves from at least one of said nares of said patient.

40. Apparatus for diagnosing the narrowing of the airways of a patient according to any one of claims 33 or 38 wherein said receiving means is at least one ultrasonic transducer located on or near to said oral mouthpiece and directed to received said reflected sound waves from said patient's oral cavity.

41. Apparatus for the detection of the occurrence of apnoea and/or hypopnoea in a patient as herein described with reference to the accompanying drawings.

42. Apparatus for the provision of CPAP treatment to the airways of a patient as herein described with reference to the accompanying drawings.

43. Apparatus for diagnosing the narrowing of the airways of a patient as herein described with reference to the accompanying drawings.
